(19) United States
(12) Patent Application Publication
(10) Pub. No.: US 2011/0054377 A1
(43) Pub. Date: Mar. 3, 2011

(54) NOSE CLEANING DEVICE

(76) Inventor: Orobola Lafe, Kent (GB)

(21) Appl. No.: 12/865,118

(22) PCT Filed: Jan. 20, 2009
(86) PCT No.: PCT/GB09/50038

§ 371 Date: Nov. 22, 2010

(30) Foreign Application Priority Data
Jan. 29, 2008 (GB) 0801607.3
Oct. 1, 2008 (GB) 0817919.4

(51) Int. Cl. A47K 7/00; A61F 13/38

(52) U.S. Cl. 604/1; 604/540

(57) ABSTRACT

A nose cleaning device comprises first and second buds, each bud at the end of a shaft, and the shafts connected together with the buds biased apart in a tweezer arrangement. The inner faces of the buds are flat, and each bud comprises a depth limiting stop for limiting the depth to which the bud can be inserted into a nostril. The invention thus provides a simple to use device which can help parents improve the comfort of their child or carers improve the comfort of disabled or elderly patients.

This invention relates to a nose cleaning device, particularly for infants and young children.

When children become congested, particularly with mucus blocking their nasal passage, they can suffer some distress at the difficulty in breathing, and also suffer further discomfort as a result of dried mucus being lodged in the nostrils. Typically, a parent will ease this distress or discomfort by helping the child to blow its nose or by attempting to clear the child's nostrils.

Attempting to help a young child to blow its nose is not always successful, as the child does not understand what is required or what is being done, and at times, blowing the nose is not sufficient enough to remove the mucus, particularly if it has dried in the nostrils.

Parents are unwilling to attempt to clear the nostrils, as there are no safe practical devices for this purpose. This invention aims to provide such a device.

It is known to use cotton buds to clear an orifice such as the ear or nose. However, there is a risk of inserting the bud too deeply, and furthermore it is not an effective tool for removing debris, particularly harder debris that does not freely adhere to the bud.

There is therefore a need for an effective device for use in clearing the nostrils.

According to the invention, there is provided a nose cleaning device comprising first and second buds, each bud at the end of a shaft, and the shafts coupled together with the buds biased apart in a tweezer arrangement, wherein the inner faces of the buds are substantially flat, and wherein each bud comprises a depth limiting stop for limiting the depth to which the bud can be inserted into a nostril.

This arrangement provides a tweezer function, but with buds which are brought apart and together so that there is no danger of the user being cut. The depth stop prevents the buds being pushed too far up the nose so that the device can be used easily and without training. The invention thus provides a simple to use device which can help parents improve the comfort of their child. The flat faces of the buds enable items to be gripped.

By “substantially flat” is meant that the inner surfaces are sufficiently flat that when brought together there is a larger area of contact than only a point contact, so that items can be gripped.

The device can be used for a child with the depth of the stops and the size of the buds being matched to the size of the child.

With the buds brought together with their flat faces in contact, they together preferably provide a smooth continuous outer profile. In other words, when brought together, the two buds can be used in the same way as a conventional single cotton bud.

The depth limiting stop can comprise a tab which projects outwardly from the base of the bud, or could be a protrusion of the bud itself.

The connected ends of the shafts can comprise a further bud (namely at the opposite end of the tweezer arrangement to the pair of buds), larger than each of the other buds. This can also be used as a normal cotton bud, and for example may be for the ears or for the nose. The further bud can also have a depth limiting arrangement.

The buds can be formed from cotton wool or other suitable soft material, and known manufacturing techniques can thus be used to form the buds. The shafts can be formed from paper, and the bud and shafts can be biodegradable. Plastic shafts can instead be used.

The shafts can comprise a grip region which comprises a locally roughened surface. This can assist in the ease of use of the device.

The device of the invention can be provided as a set, with different devices for a different age or size of person, with each nose cleaning device having the depth limiting stops at different depths, and the buds of the appropriate size.

An example of the invention will now be described in detail with reference to the accompanying drawings, in which:

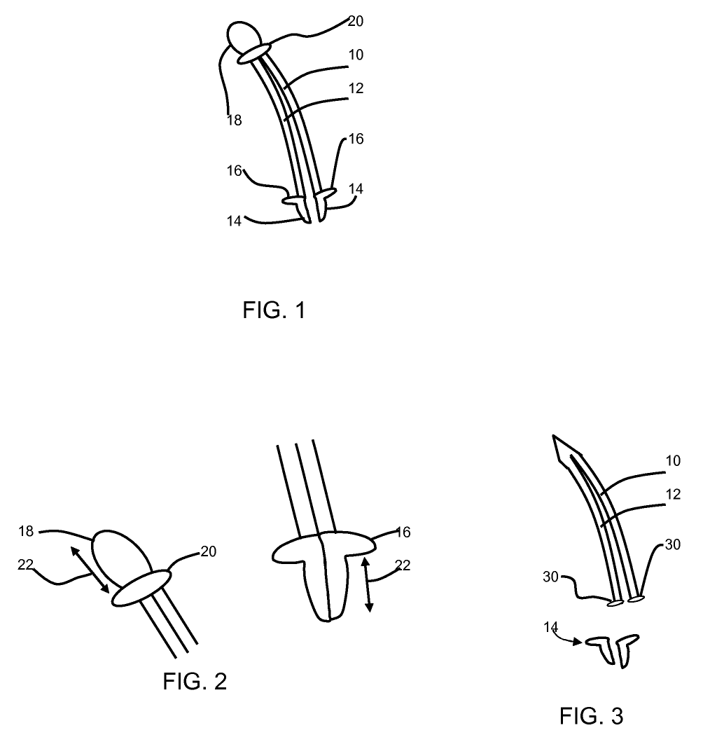

FIG. 1 shows a first example of nose cleaning device of the invention;

FIG. 2 shows the bud parts of FIG. 1 in greater detail, and with the pair of buds brought together;

FIG. 3 shows a second example of nose cleaning device of the invention.

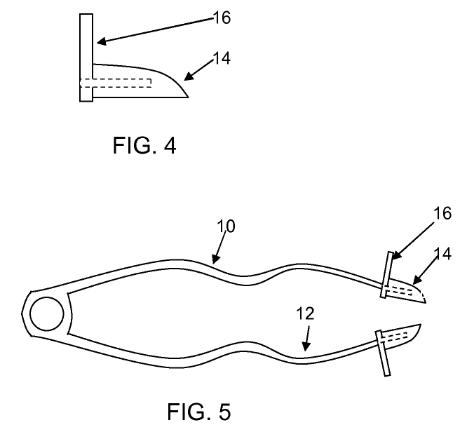

FIG. 4 shows another example of bud part; and

FIG. 5 shows a third example of nose cleaning device of the invention using the bud design of FIG. 4.

FIG. 1 shows a nose cleaning device of the invention which is generally in the form of a pair of tweezers. Each tweezer arm 10,12 has a cotton bud 14 (or other absorbent material bud) at the end. The tweezer arms are formed as shafts over which the buds are mounted. The buds thus define enlarged head regions supported by the shafts.

The shafts 10,12 are connected together with the buds biased apart as shown in FIG. 1. This bias of the shafts apart can be achieved in a number of ways. The shafts need to have sufficient rigidity and elasticity that they will hold their form, and can be deformed and will return to their original form. Plastic or paper shafts are suitable for this purpose.

The separation of the shafts can be by mounting a spacer near the connected end. It is also possible to use a stronger “V” shaped element at the joined ends, to provide the bias into the opened state. However, it is preferred that the structure is fully made from paper, so that the device is recyclable and/or biodegradable.

The device is preferably designed as a single use disposable item, and is not designed to be cleaned during or after use.

The inner faces of the pair of buds are flat so that mucus or other nasal deposits can be gripped and extracted. The cotton buds can be formed over a suitably shaped shaft end to create the desired shape. Each bud also comprises a depth limiting stop 16 for limiting the depth to which the bud can be inserted into a nostril. In this example, the depth limiting stop 16 is in the form of a tab which projects outwardly from the base of the bud, however, the stop could be an integral protrusion from the bud itself. The size of the tab is such that the total width of the two tabs and two buds is larger than the nostril opening of a size of child for whom the device is intended.

Different versions of the device can have different bud sizes and different distances from the end of the buds to the stop 16, so that a device is used which is suitable for a particular size of child.

With the buds brought together with their flat faces in contact, they together provide a smooth continuous outer profile. This is shown in FIG. 2, which also shows the depth dimension 22. This dimension will typically be in the range 3 mm to 12 mm. When the buds are brought together, the two buds can be used in the same way as a conventional single cotton bud.

In the example shown, the connected ends of the shafts share a further bud 18. This is larger than each of the other buds, but may have a size corresponding to the combined size of the pair of buds. This can also be used as a normal cotton bud, and also has a depth limiting arrangement 20 in the form of tabs or a continuous ring around the base.

The further bud can thus also be for the nose. For example, the pair of buds are used for removing material, for example hardened mucus, and the single bud is used for subsequent cleaning. This avoids the need to clean the pair of buds as a clean bud is already available for a final nasal cleaning operation.

The further bud may however be of different design and for a different purpose, for example for the ear.

The shafts can comprise a grip region (not shown) which comprises a locally roughened surface. This can assist in the ease of use of the device.

The buds can be sold as a set of different sizes, each for a different age or size of person, with each nose cleaning device having a different depth limiting stop distance 22 and different sized buds.

The buds can be made from cotton or other cellulose-based material, although synthetic materials can also be used. The fibers used may be absorbent and/or the open spaces used between fibers within the structure can be used as the mechanism for absorbing and cleaning.

In the example above, the buds are formed on the tweezer arms during manufacture, and the finished product is supplied to the user in the form shown in FIG. 1. In an alternative arrangement, the buds are removable from the tweezer arms, for example being fitted to the ends of the tweezer arms as a snap fit. A set can then comprise a smaller number of tweezer parts (possibly only one tweezer part, or a small number so that there are some spares) and a much larger number of buds which can be attached.

The buds are primarily designed for their gripping function rather than for absorbency. The buds may for example comprise soft rubber or silicone. The tweezer part may be recyclable plastic or metal.

FIG. 3 shows an example of this approach, using the same reference numbers as in FIGS. 1 and 2. The buds 14 are shown as separate from the tweezer, and a clip 30 is shown schematically at the end of the tweezer arms. This provides a snap fit with an internal feature of the bud, so that the buds are a secure snap fit onto the ends of the tweezer arms. Any suitable retaining feature can be used for this purpose.

FIG. 4 shows another possible design of bud 14. The part that enters the nostril is substantially conical (but with a flatter inner face), so that it has a progressively larger diameter. This shape has a self-limiting feature, so that it can only be inserted a certain distance depending on the size of the nostril. There is also the stop 16 to provide a final limit to the insertion depth. The stop is an integral part of the bud 14 and can comprise a disc from which the bud part projects. A passageway extends along the inside of the conical part for gripping the tweezer end.

The design of the bud in FIG. 4 is such that a single size bud can be used for different ages or nostril sizes, thereby eliminating the need for different size buds. Alternatively, different bud sizes could be made for different ages or nostril sizes if desired.

FIG. 5 shows the bud design of FIG. 4 clipped onto the end of a different tweezer design. This is a moulded one-piece plastics tweezer part 10,12 with the hinge defined as an integral part of the plastics component. The middle of each part 10,12 has a recess for the user to grip.

The description above discusses the use of the device for infants. However, the device is equally applicable for mentally or physically handicapped patients and elderly individuals who need care, and thus adult size devices are also appropriate.

In one version, the invention essentially provides a re-engineered cotton bud which is specifically of use for the nostrils. However, the invention can be formed using many different materials as outlined above.

Various modifications will be apparent to those skilled in the art.

1. A nose cleaning device comprising first and second buds, each bud at the end of a shaft, and the shafts coupled together with the buds biased apart in a tweezer arrangement, wherein the inner faces of the buds are substantially flat, and wherein each bud comprises a depth limiting stop for limiting the depth to which the bud can be inserted into a nostril.

2. A device as claimed in claim 1, wherein with the buds brought together with their flat faces in contact, they together provide a smooth continuous outer profile.

3. A device as claimed in any preceding claim, wherein the depth limiting stop comprises a tab or protrusion which projects outwardly from the base of the bud.

4. A device as claimed in any preceding claim, wherein the connected ends of the shafts comprise a further bud, larger than each of the other buds.

5. A device as claimed in claim 4, wherein the further bud also has a depth limiting arrangement.

6. A device as claimed in any preceding claim, wherein the buds are removable from the shaft ends.

7. A device as claimed in claim 6, wherein the buds are a snap fit on the shaft ends.

8. A device as claimed in any preceding claim, wherein the buds are formed from cotton wool or other absorbent material.

9. A device as claimed in any one of claims 1 to 7, wherein the buds are formed from a rubber material.

10. A device as claimed in claim 9, wherein the buds have a conical shape with a flattened side face.

11. A device as claimed in any preceding claim, wherein the shafts are formed from paper, plastic or other suitable recyclable or bio-degradable products.

12. A device as claimed in any preceding claim, wherein the shafts comprise a grip region consisting of a locally roughened surface.

13. A set of nose cleaning devices, each for a different age or size of person, each nose cleaning device as claimed in any preceding claim, with each nose cleaning device having the depth limiting stops at different depths and different size buds.
